SEQUENCE LISTING (8 sequences)

SEQ ID NO: 1 - DNA, 20 bp, synthetic construct
  source 1..20: mol_type = other DNA
cggctaccacatccaaggaa

SEQ ID NO: 2 - DNA, 18 bp, synthetic construct
  source 1..18: mol_type = other DNA
gctggaattaccgcggct

SEQ ID NO: 3 - DNA, 20 bp, synthetic construct
  source 1..20: mol_type = other DNA
gctagcagcctctctctcct

SEQ ID NO: 4 - DNA, 20 bp, synthetic construct
  source 1..20: mol_type = other DNA
gtgggatgtgccatcaacct

SEQ ID NO: 5 - DNA, 20 bp, synthetic construct
  source 1..20: mol_type = other DNA
ggaagacccctcccagatag

SEQ ID NO: 6 - DNA, 20 bp, synthetic construct
  source 1..20: mol_type = other DNA
ggaagacccctcccagatag

SEQ ID NO: 7 - DNA, 20 bp, synthetic construct
  source 1..20: mol_type = other DNA
tgaaagctctccacctccag

SEQ ID NO: 8 - DNA, 20 bp, synthetic construct
  source 1..20: mol_type = other DNA
cacgcaggacaggtacagat

(19) United States
(12) Patent Application Publication
(10) Pub. No.: US 2026/0014132 A1
(43) Pub. Date: Jan. 15, 2026

(54) PROTEASE ACTIVATED RECEPTOR 2 (PAR2) INHIBITOR AND ITS USES

(71) Applicant: UNIVERSITÀ DEGLI STUDI DI PADOVA, Padova PD (IT)

(72) Inventors: Patrizia Pontisso, Padova PD (IT); Alessandra Biasiolo, Padova PD (IT); Laura Cendron, Padova PD (IT); Monica Chinellato, Padova PD (IT); Santina Quarta, Padova PD (IT); Mariagrazia Ruvoletto, Padova PD (IT); Gianmarco Villano, Padova PD (IT); Cristian Turato, Padova PD (IT)

(73) Assignee: UNIVERSITÀ DEGLI STUDI DI PADOVA, Padova PD (IT)

(21) Appl. No.: 18/992,537

(22) PCT Filed: Jul. 12, 2023
(86) PCT No.: PCT/IB2023/057138

§ 371 Date: Jan. 8, 2025

(30) Foreign Application Priority Data
Jul. 12, 2022 (IT) 102022000014593

(51) Int. Cl. A61K 31/445; A61P 37/06

(52) U.S. Cl. CPC A61K 31/445; A61P 37/06

(57) ABSTRACT

The present invention relates to a PAR2 inhibitor, in particular to 1-piperidine propionic acid or to pharmaceutical compositions comprising it, for use in the treatment or prevention or to assist the treatment or prevention of PAR2-related pathological conditions.

The present invention relates to a PAR2 inhibitor, in particular to 1-piperidine propionic acid or to pharmaceutical compositions comprising it, for use in the treatment or prevention or in assisting the treatment or prevention of pathological conditions related to PAR2 or to therapeutic methods for the treatment of such pathological conditions, comprising the administration of 1-piperidine propionic acid or pharmaceutical compositions comprising it.

### STATE OF ART

Protease-activated receptor 2 (PAR2) is a cell surface protein related to G-protein-dependent and independent intracellular signaling pathways that produce a broad range of physiological responses, including those related to metabolism, inflammation, pain, and cancer. Potent PAR2 activators are known such as, proteases, peptides, and non-peptides. However, effective PAR2 antagonists have not yet been reported in the literature despite their anticipated therapeutic potential.

It is known in the literature that PAR2 overexpression is related to numerous diseases such as, for example, rheumatoid arthritis, graft versus host disease (GVHD), respiratory distress syndrome (ARDS), sepsis, septic shock, Ebola, influenza avian flu, COVID-19 disease, smallpox, MERS, SARS, systemic inflammatory response syndrome, hantavirus pulmonary syndrome, lymphohistiocytosis haemophagocytic disease, tumors, including tumors that do not overexpress SerpinB3, endothelial dysfunction, acute lung injury (ALI, Acute Lung Injury), neurodegenerative diseases, osteoarthritis, post-traumatic osteoarthritis, Chron, Alzheimer, Parkinson, Multiple Sclerosis, ALS, thrombosis caused by an inflammatory event, inflammatory pain, edema, asthma, respiratory allergies, post-surgery pain, hyperalgesia, pain neuropathic, fracture pain, osteoporotic fracture pain, bone cancer pain or gout joint pain, irritable bowel syndrome pain, inflammatory bowel pain, ulcerative colitis, cancer, and also resistance to some cancers, such as pulmonary diseases, treatments with EFGR inhibitors, inflammatory brain diseases, pancreatitis, asthma, metabolic heart disease, atherosclerosis, proliferative retinitis, arterial vasodilatation, visceral pain, neoplastic pain, migraine, cancer pain (Jiang et al 2021 frontieres in Pharmacology 1-11 “Targeting PAR2 overcomes Geftinib resistance in Non-Small-Cell Lung cancer cells attraverso inhibition of EGFR transactivation”; Mcintosh et al 2020 Biochemical Society Transactions Vol 48 pp 2525-2537 “The development of proteinase-activated receptor-2 modulators and the challenges involved”; Cheng et al 2017 Nature Vol 545 pp 112-115” Structural insight into allosteric modulation of protease-activated receptor-2).

The correlation between PAR2 activation/overexpression with a large number of pathological conditions is widely supported in the literature and numerous studies have been published aimed at identifying effective inhibitors of this receptor for therapeutic purposes.

However, although PAR2 is a therapeutic target of great interest, there is currently no clinically authorized PAR2 inhibitor.

The identification, preferably among known molecules, safe for human use, of effective inhibitors of PAR2 in order to be able to use them for therapeutic use is therefore of great interest.

### SUMMARY OF THE INVENTION

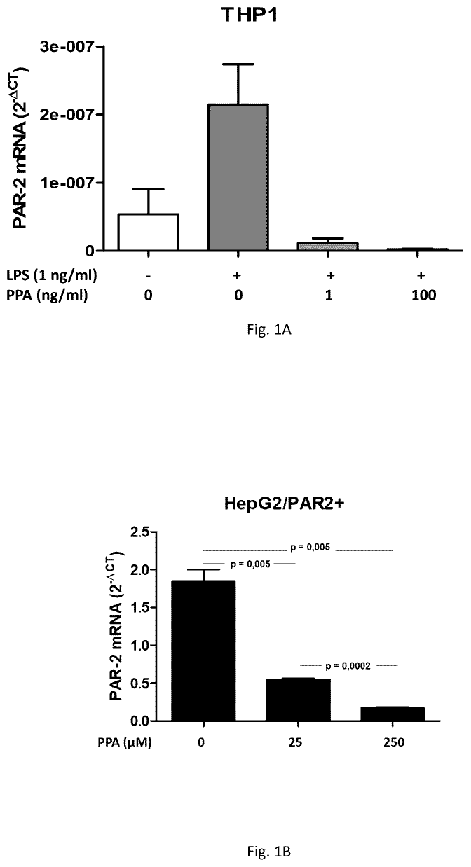

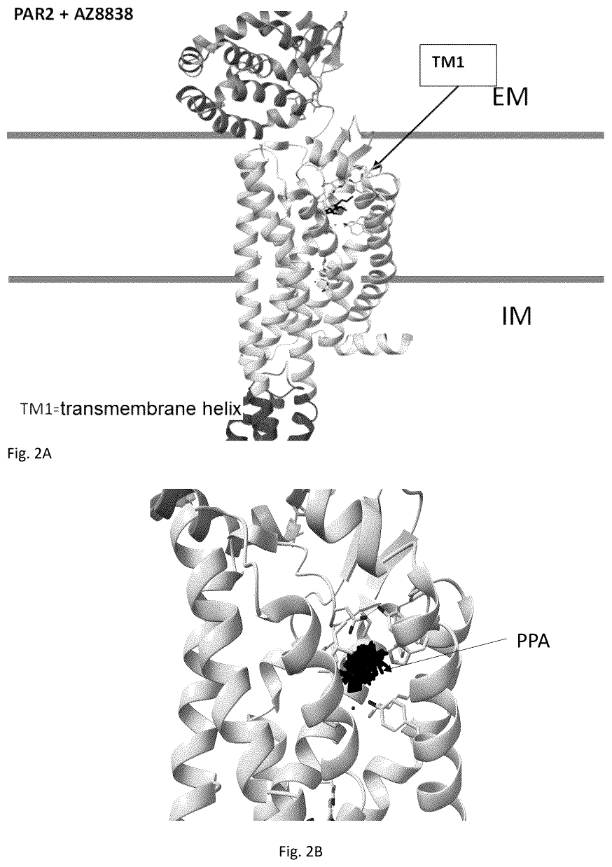

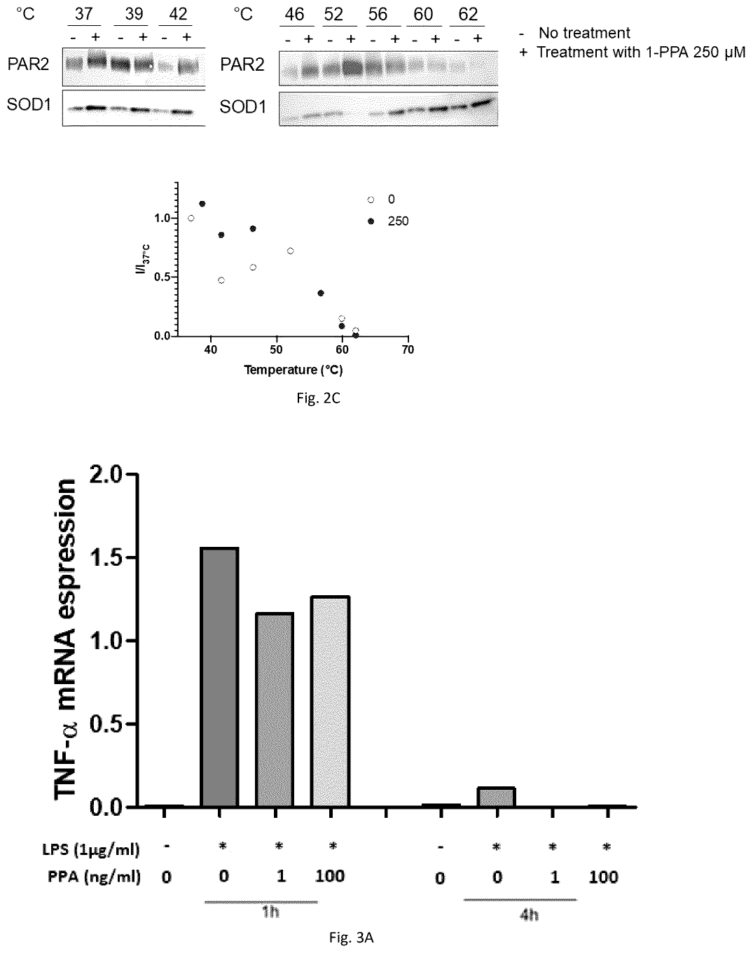

The authors of the present invention have surprisingly found that 1-piperidine propionic acid (1-PPA) or a salt thereof is an effective inhibitor of PAR2, both at the transcriptional level (FIG. 1) and at the functional level (FIG. 2). In fact, the authors used 1-PPA on THP-1 cells in which the synthesis of PAR2 had been induced, even at very low concentrations, in the order of 1 ng/ml, and discovered that this molecule strongly inhibits the transcription of the receptor membrane membrane PAR2, in particular PAR2 RNA synthesis is inhibited by 1-piperidine propionic acid in a dose-dependent manner, as demonstrated in FIG. 1A. In the figure, in fact, it is shown that the expression of PAR2, significantly increased after incubation with lipopolysaccharide (LPS), is drastically reduced upon the addition of 1-PPA, suggesting that the biological action of 1-PPA occurs at the transcriptional level, particularly through inhibition of PAR2 membrane receptor synthesis. Analogous results were also obtained in HepG2 cells genetically modified to overexpress PAR2 (FIG. 1B), where the transcriptional expression of PAR2 is reduced by 1-PPA in a dose-dependent manner.

The authors also verified that the same molecule is also a functional PAR2 inhibitor by comparing the interaction site of a known PAR2 antagonist reported in the literature with said receptor, and the interaction site of 1-PPA, using molecular docking computational models with said receptor. This comparison resulted in a coincidence between the interaction site of the known antagonist and the interaction site of 1-PPA with the PAR2 receptor.

As regards the structural level, in fact, FIG. 2 shows how, following the evaluation of the possible steric interaction between PAR2 and 1-PPA, it resulted that 1-PPA is positioned in the space between the transmembrane helices of PAR2 and interacts mainly with helices 1-2-7, acting as an antagonist, exactly like the compound AZ8838 (Cheng et al 2017) which is a known PAR2 inhibitor and was therefore used as a control. These interaction data between 1-PPA and PAR2 were also confirmed at the protein level, using the cellular thermal shift assay (CETSA) (Science 341 (6141): 84-87). This is a biophysical assay based on the principle of ligand-induced thermal stabilization of target proteins, as the melting temperature of proteins changes according to the interaction of the ligand. FIG. 2C shows how in the course of a progressive increase in temperature, the presence of 1-PPA determines a stabilization of PAR2 in HepG2 cell lysates transfected to overexpress PAR2.

Furthermore, 1-PPA significantly reduces the synthesis of LPS-induced inflammatory cytokines involved in cytokine storm, reasonably both through steric receptor blockade, as 1-PPA positions itself precisely at the binding site of a known PAR2 inhibitor, AZ8838, which by transcriptional inhibition of the PAR2 membrane receptor.

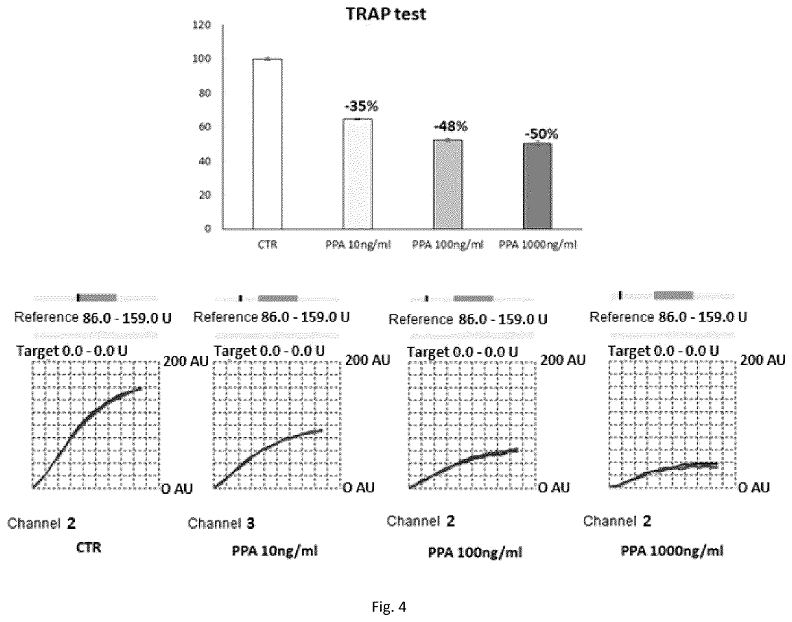

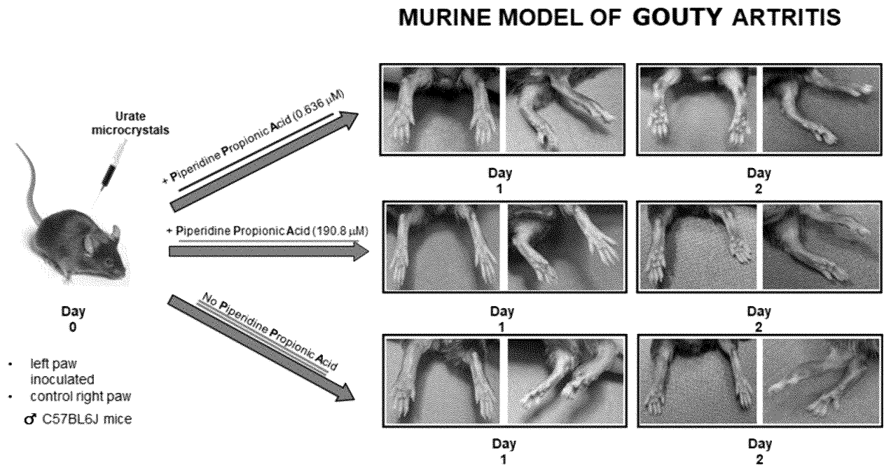

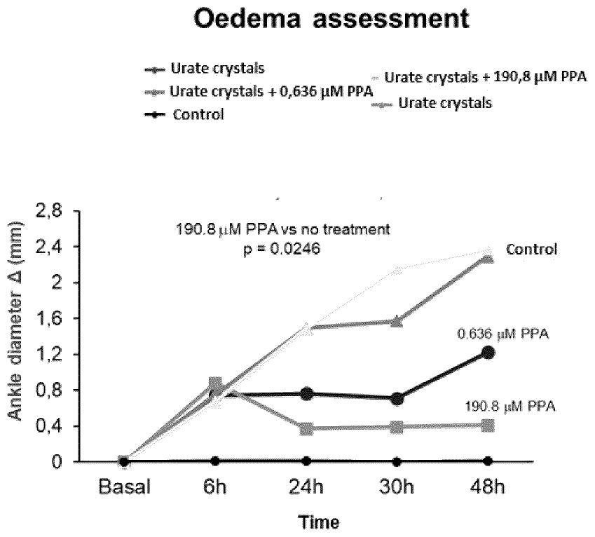

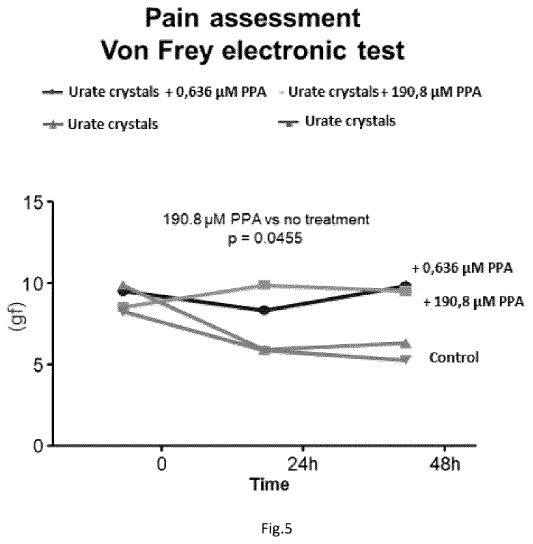

1-PPA has been shown to reduce platelet aggregation induced by PAR2 activation in a dose-dependent manner (FIG. 4). As clearly demonstrated by the results of the in vivo tests conducted by the inventors and reported in the experimental section of the present description, 1-piperidine propionic acid is capable of exerting a protective effect in a mouse model of gouty arthritis, significantly reducing not only the inflammation, but also joint pain (FIG. 5).

For example, 1-piperidine propionic acid significantly reduces in monocytes the synthesis of inflammatory cytokines known to be induced by PAR2 overexpression, which, as evident from FIG. 1, induces PAR2 expression, such as TNF-α and Interleukin 1b (IL1-β). The reported data show how this molecule, by inhibiting the expression of PAR2, in turn inhibits the expression of the aforementioned cytokines.

Therefore, the use of 1-piperidine propionic acid as an effective inhibitor of PAR2, both at the transcriptional and at the functional level, is particularly indicated for the treatment or prevention or to assist the treatment or prevention of pathological conditions related to PAR2.

In particular, to pathological conditions caused or co-caused by the overexpression or by a positive modulation of PAR2.

By way of example, 1-piperidine propionic acid can be used in the treatment, or to assist the treatment, of one or more of the following pathological conditions; graft versus host disease (GVHD), respiratory distress syndrome (ARDS), sepsis, septic shock, Ebola, avian influenza, COVID-19 disease, smallpox, MERS, SARS, systemic inflammatory response syndrome, hantavirus pulmonary syndrome, lymphohistiocytosis hemophagocytic disease, tumors that do not overexpress SerpinB3, rheumatoid and inflammatory arthritis, osteoarthritis, posttraumatic osteoarthritis, endothelial dysfunction, acute lung injury (ALI, Acute Lung Injury), neurodegenerative diseases, Chron, Alzheimer, Parkinson, Multiple Sclerosis, ALS, thrombosis caused by an inflammatory event, hyperalgesia, edema, asthma, respiratory allergies, inflammatory pain, post-surgery pain, neuropathic pain, fracture pain, osteoporotic fracture pain, bone cancer pain or joint pain from gout, irritable bowel syndrome pain, inflammatory bowel pain, ulcerative colitis, cancer, tumors resistant to treatment with EGFR inhibitors, inflammatory brain disease, pancreatitis, asthma, metabolic heart disease, atherosclerosis, proliferative retinitis, arterial vasodilatation, visceral pain, neoplastic pain, migraine, cancer pain.

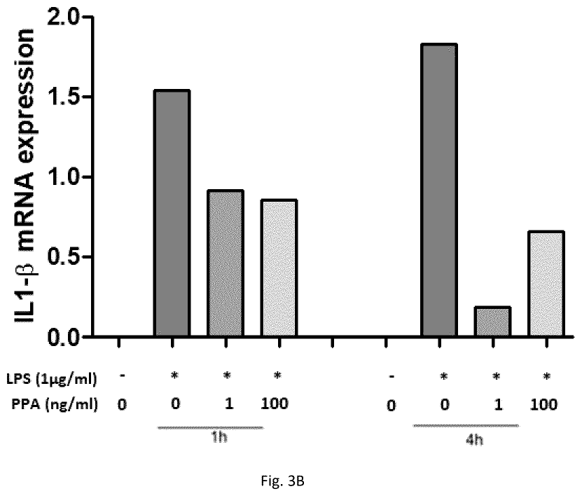

For example, since it is reported in the literature that PAR2 appears to be involved in thrombosis episodes in patients with a SARS-Cov-2 infection (Figure 3 of Subramaniam et al. “Advocacy of targeting protease-activated receptors in severe coronavirus disease 2019”, Br J Pharmacol 2021; 1-14), 1-PPA acid can be used as an antithrombotic, in cases where thrombosis is caused by an inflammatory problem.

Further, 1-PPA can be used as a pain reliever by acting as a PAR2 inhibitor, since PAR2 is involved in pain perception mechanisms making nerve endings highly sensitive to painful stimuli, as reported in the article by Mrozkova et al. “The Role of Protease-Activated Receptor Type 2 in Nociceptive Signaling and Pain.” 2016, Physiol. Res. 65; 357-367).

Furthermore, 1-PPA can be used in the treatment of tumors, including tumors that are resistant to EGFR inhibitors (such as gefitinib).

Furthermore, 1-PPA can be used in the treatment of various forms of arthritis, viral infections and neurodegenerative diseases.

Advantageously, 1-piperidine propionic acid is easily commercially available, is well tolerated in experimental models, is used in cosmetics for human use, is difficult to degrade and has a low cost.

Therefore, objects of the present invention are

A protease activated receptor 2 (PAR2) inhibitor for use in the treatment or prevention of or to assist in the treatment or prevention of related pathological conditions said receptor wherein said inhibitor is 1-piperidine propionic acid or a salt thereof.

1-piperidine propionic acid or a salt thereof for use in the treatment or prevention of, or to aid in the treatment or prevention of PAR2-related pathological conditions;
a pharmaceutical composition comprising 1-piperidine propionic acid or a salt thereof and at least one pharmaceutically acceptable carrier for use in the treatment or prevention or to assist in the treatment or prevention of PAR2-related pathological conditions;
the use of 1-piperidine propionic acid or a salt thereof as a PAR2 inhibitor
the use of 1-piperidine propionic acid as a PAR2 inhibitory active ingredient in the preparation of pharmaceutical or cosmetic compositions.

In jurisdictions where this is permitted, the invention also relates to a method for treating or preventing, or to assisting in the treatment or prevention of PAR2-related pathological conditions, which comprises the administration of 1-piperidine propionic acid or a pharmaceutical composition comprising it in a therapeutically effective dosage to a subject in need of it.

### Glossary

PAR2 according to the present description has the meaning commonly known in the literature and therefore refers to the receptor activated by protease 2 as also reported in the prior art documents cited above.

PAR2 also known as coagulation factor II (thrombin) receptor (F2RL1) or G protein-coupled receptor 11 (GPR11) is a protein that in humans is encoded by the F2RL1 gene. PAR2 modulates inflammatory responses, obesity, metabolism, tumors and acts as a sensor for proteolytic enzymes generated during infections. In humans, PAR2 can be found in the stratum granulosum of epidermal keratinocytes. Functional PAR2 is also expressed by various immune cells such as eosinophils, neutrophils, monocytes, macrophages, dendritic cells, mast cells and T cells.

The expression PAR2-related pathological conditions in the present description refers to all those pathological conditions which benefit from the inhibition of the synthesis and/or activity of the membrane receptor of PAR2. These pathological conditions therefore also include pathological conditions caused or co-caused by the overexpression or by a positive modulation of PAR2.

1-Piperidine Propionic Acid (herein also referred to as 1-PPA) (CAS Registry Number: 26371-07-3) also called 1-Piperidine-propanoic acid or 3-(1-Piperidinyl) propionic acid, or piperidine propionic acid is an amino acid of the formula:

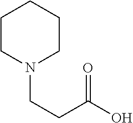 [SMILES: O=C(O)CCN1CCCCC1]

Lipopolysaccharide (LPS): component of the cell membrane of Gram-negative bacteria, consisting of three main regions: lipid A, or the glycolipidic portion; an oligosaccharide core; the O antigen, i.e. a polysaccharide chain consisting of 20-50 repeating units which generally can contain up to eight sugars.

In any point of the description and of the claims the term 1-piperidine propionic acid (1-PPA) is intended as 1-piperidine propionic acid or a salt thereof or mixtures of its salts or mixtures of 1-piperidine propionic acid and one or more thereof salts, in particular pharmaceutically acceptable salts.

Furthermore, at any point in the description and in the claims the term comprising may be replaced by the term consisting of.

By expression tumors that do not overexpress SerpinB3, we mean tumors in which the expression of SerpinB3 is not increased (upregulated) compared to the normal expression of said protein in healthy tissue or in healthy controls.

### DETAILED DESCRIPTION OF THE FIGURES

FIG. 1 shows the evolution of PAR2 mRNA expression induced by LPS, following addition of 1-PPA in a human monocyte cell line (THP1) and transfected HepG2 cells transiently to overexpress PAR2. The results demonstrate that PAR2 transcription is dose-dependently inhibited by 1-PPA. As described in Example 1A, the expression of PAR2 was found to be markedly increased after incubation with LPS, and the addition of 1-PPA induced a marked reduction of its expression, suggesting that the biological action of this compound occurs at transcriptional level, in particular through the inhibition of the synthesis of the PAR2 membrane receptor. Analogous results were also obtained in cells overexpressing PAR2, as described in Example 1B.

FIG. 2A shows the steric interaction between PAR2 and its known inhibitor (AZ8838) used as a model for the Molecular Docking shown.

FIG. 2B shows the steric interaction between PAR2 and 1-PPA, and that PAR2 and 1-PPA interact in a similar manner to the pair PAR2 and AZ8838, using a conformation identified as level 0 classes.

FIG. 2C shows the result of the interaction between 1-PPA and PAR2 at the protein level, where, using the cellular thermal shift assay (CETSA), a thermal stabilization of PAR2 is documented in the presence of 1-PPA.

FIG. 3A shows the expression trend of the inflammatory cytokine TNF-α, induced by the addition of LPS, in the THP-1 cell line of monocyte origin; the synthesis of pro-inflammatory cytokines is effectively blocked by the addition of 1-PPA.

FIG. 3B shows the trend of the expression of the inflammatory cytokine IL 1-β, induced by the addition of LPS, in the THP-1 cell line of monocyte origin; the synthesis of pro-inflammatory cytokines is effectively blocked by the addition of 1-PPA.

FIG. 4 shows the ability of 1-PPA to inhibit platelet aggregation in a dose-dependent manner, with excellent reproducibility in the experiments performed, demonstrating that 1-PPA is capable of inhibiting the pro-coagulant activity induced by PAR2.

FIG. 5 shows the protective effect of 1-PPA in a mouse model of gouty arthritis. The figure shows that the administration of 1-PPA significantly reduces the edema and inflammation induced by the injection of urate microcrystals, and in particular, that it also reduces the paw pain perceived by the animal and measured by the Von Frey electronic test.

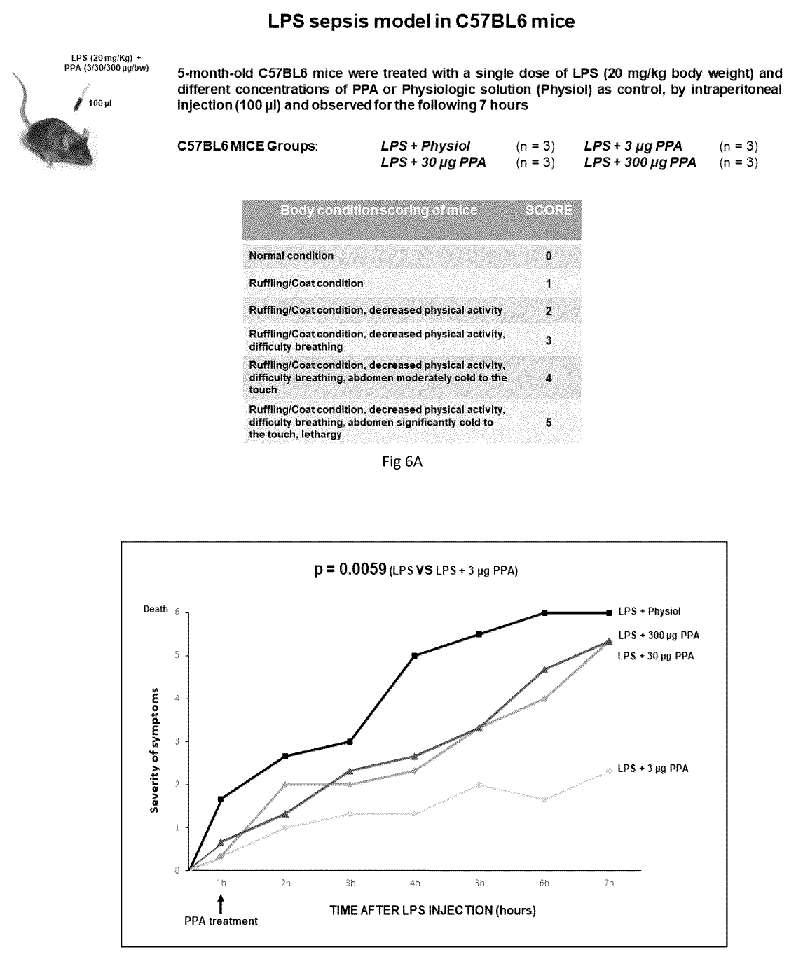

FIG. 6A shows that low concentrations of 1-PPA protect the mouse from a model of LPS-induced sepsis, which according to the illustrated scale results in the death of the animal within 6 hours. The lowest concentration of 1-PPA (3 μg) in fact documents how a maximum of a score 2 is reached which highlights a slight suffering, with no respiratory effort in the animal, while untreated animals progressively worsen and die.

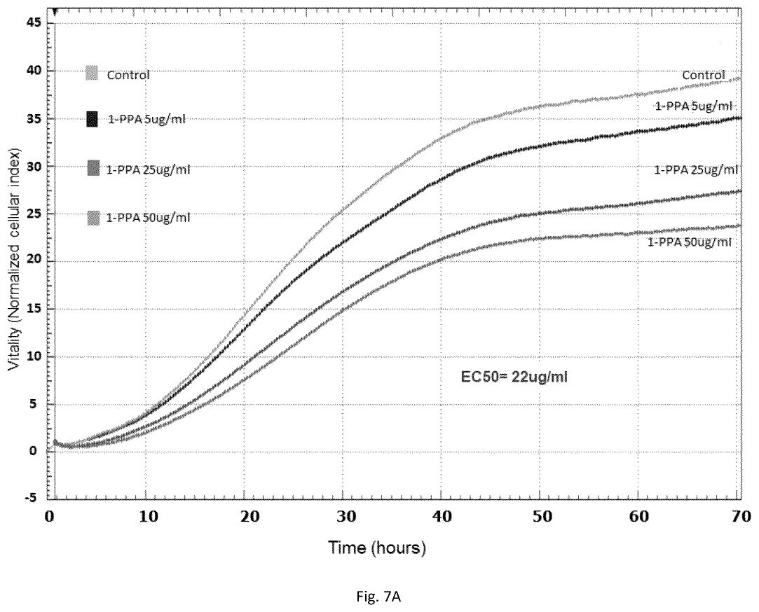

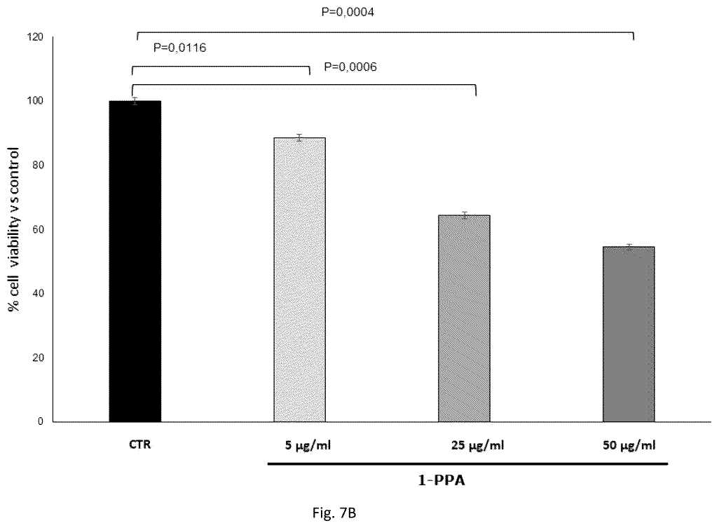

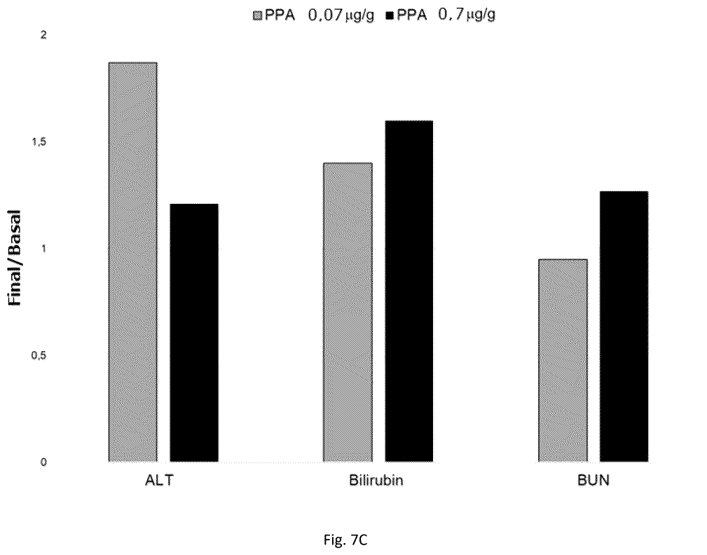

FIG. 7 shows the results of 1-PPA toxicity studies. In in vitro experiments, using the HepG2 cell line (code HB8065), increasing concentrations of the compound 1-PPA (5-50 μg/ml) did not demonstrate a cellular toxic effect at the concentration of 5 μg/ml, which is at least 50 times the biologically effective dose. (FIG. 7A, 7B).

FIG. 7C reports the results obtained in the in vivo experiments, in which 1-PPA was found to be scarcely toxic at both the hepatic and renal level.

FIG. 7C shows, in particular, that in CC57/BL6 mice inoculated with 1-PPA at a concentration equal to 0.07 μg/g/week and at a 10 times higher concentration equal to 0.7 μg/g/week, the level of alanine amino transferase (ALT), used as an index of cytolysis, nor of bilirubin, used as an index of cholestasis, nor the levels of blood urea nitrogen, an expression of renal function. (BUN) Blood Urea Nitrogen viz urea nitrogen,

### DETAILED DESCRIPTION OF THE SEQUENCES

45S gene sense primer
SEQ ID 1
5′-CGGCTACCACCATCCAAGGAA-3′
45S gene antisense primer
SEQ ID 2
5′-GCTGGAATTACCGCGGCT-3′
PAR2 gene sense primer
SEQ ID 3
5′-GCTAGCAGCCTCTCTCTCCT-3′
PAR2 gene antisense primer
SEQ ID 4
5′-GTGGGATGTGCCATCAACCT-3′
TNF-α gene sense primer
SEQ ID 5
5′-AACCTCCTCTCTGCCATCAA-3′
TNF-α gene antisense primer
SEQ ID 6
5′-GGAAGACCCCTCCCAGATAG-3′
IL-1β gene sense primer
SEQ ID 7
5′-TGAAAGCTCTCCACCTCCAG-3′
IL-1β gene antisense primer
SEQ ID 8
5′-CACGCAGGACAGGTACAGAT-3′

### DETAILED DESCRIPTION OF THE INVENTION

A first aspect of the present invention provides an active agent, 1-piperidine propionic acid for use in the treatment or prevention or to assist in the treatment or prevention of PAR2-related pathological conditions.

As reported in Example 1, the authors of the invention discovered and demonstrated that 1-piperidine propionic acid is particularly effective in significantly reducing PAR2 levels, both induced by LPS and after gene overexpression using an expression plasmid of PAR2, demonstrating, in fact, that 1-PPA is certainly an inhibitor of PAR2 at the transcriptional level, i.e. it inhibits the transcription of the PAR2 gene into mRNA.

Furthermore, the authors of the invention have also shown the correspondence between the binding site of functional inhibitors of PAR2, in particular the binding site described for the inhibitor AZ8838 (Cheng et al 2017) corresponds to the one resulting from molecular docking for 1-PPA and is a small binding pocket delineated between residues from TM1-3, TM7 and ECL2 of PAR2 (see figures 1 and 2a of Cheng et al 2017. Figure 3 of Cheng et al 2017 accurately reports the inhibition detected by FLIPR by the antagonist AZ8838) thus demonstrating that 1-PPA is also a functional inhibitor of PAR2, or in other words, an antagonist of the molecules that activate PAR2.

Furthermore, the steric interaction between PAR2 and 1-PPA was also confirmed at the protein level as shown in FIG. 2C where the CESTA method was used. PAR2 inhibition, for the purposes of the present invention, can also be measured by Ca2+ ion flux analysis or even by beta-arrestin recruitment assays.

Non-limiting examples of PAR2-related pathological conditions include: graft versus host disease (GVHD), respiratory distress syndrome (ARDS), sepsis, septic shock, Ebola, avian influenza, COVID-19 disease, smallpox, MERS, SARS, systemic inflammatory response syndrome, hantavirus pulmonary syndrome, lymphohistiocytosis haemophagocytic disease, tumors that do not overexpress Serpin B3, rheumatoid and inflammatory arthritis, osteoarthritis, posttraumatic osteoarthritis, endothelial dysfunction, acute lung injury (ALI, Acute Lung Injury), Chron's disease, neurodegenerative diseases, Alzheimer's, Parkinson's, Multiple Sclerosis, ALS, thrombosis caused by an inflammatory event, hyperalgesia, edema, asthma, respiratory allergies, inflammatory pain, post-surgery pain, neuropathic pain, joint pain fracture, osteoporotic fracture pain, bone cancer pain or gout joint pain, irritable bowel syndrome pain, inflammatory bowel pain, ulcerative colitis, cancer, tumors resistant to treatment with EGFR inhibitors, inflammatory brain disease, pancreatitis, asthma, metabolic heart disease, atherosclerosis, proliferative retinitis, arterial vasodilatation, visceral pain, neoplastic pain, migraine, cancer pain, but are not limited to them. The data obtained by the inventors show how 1-PPA, administered parenterally even in minimal doses, is capable of inhibiting PAR2 transcription in cell lines in which said transcription is induced with LPS or by gene transcription using expression plasmids.

Numerous pathologies in which the inhibition of PAR2 has a therapeutic effect are described in the literature, the table below shows a non-exhaustive but indicative list of such pathologies whose treatment by administration of 1-PPA falls within the object of the present invention

| Pathology | Inhibitor | Reference |
|---|---|---|
| Arthritis | Anti-PAR2 mabs | Amiable et al J Rheumatol 2011, 38: 911-920, Ferrel et al |
| | PAR2 siRNA | Ann Rheum Dis 2010, 69: 2051-2054 |
| | Pepducin | Xue et al, Arthritis Rheum 2012, 64: 88-98 |
| | | Sevigni et al PNAS 2011 |
| | | www.pnas.org/cgi/doi/10.1073/pnas.1017091108 |
| Colitis | PAR2 Knock Out mice | Hyun et al Inflamm Res 2010, 59: 699-709 |
| Irritable bowel | GB88 | Lohman et al J Pharmacol exp Ther 2012, 340: 256-265 |
| | PAR2 antagonist | Jimenez-Vargas, PNAS 2018, |
| | | www.pnas.org/cgi/doi/10.1073/pnas.1721891115 |
| Alzheimers | PAR2 KO mice | Afkhami-Goli et al 2007, doi: |
| Parkinsons | IHC human brains | 10.4049/jimmunol.179.8.5493 |
| Cerebral inflammatory | Review | Hurley et al J Mol Neurosci 2015 DOI 10.1007/s12031- |
| pathologies | | 015-0576-8 |
| | | Bushell et al Curr Drug Targets 2016 DOI: |
| Pancreatitis | Review | Suhaj et al Phsiol Res 2022, |
| | | https://doi.org/10.33549/physiolres.934931 |
| Asthma | PAR2 Knock Out mice | Page et al, Respir Res 2010, 11: 62 de Boer et al Inn Immun |
| | | 2014, 20: 618-625 |
| Neoplastic pain | PAR2 Knock Out mice | Lam et al J Neurosci 2012, 32: 14178-183 |
| Pain/Hyperalgesia | PAR2 Knock Out mice | Zhao et al J Biol Chem 2015, 290: 1375-64, Liu et al Eur J |
| Ache | Review | Pain 2014, 18: 326-337 |
| Migraine | Anti-PAR2 mabs | Mrozkova et al |
| Visceral pain | (PAR650097) | physiol Res 2016 |
| | Review | https://doi.org/10.33549/physiolres.933269 |
| | | Kopruszinski et al, Chephalgia 2020, DOI: |
| | | 10.1177/0333102420966581 |
| | | Cenac N. Curr Neuropharmacol 2013, 11: 598-605 |
| Metabolic heart disease | Review | Jones et al. Arter Thromb tank Biol 208 DOI: |
| Atherosclerosis | PAR2 Knock Out mice | 10.1161/ATVBAHA.117.310082 |
| Proliferative retinitis | PAR2 Knock Out mice | Joyal et al Nat Med 2014, 20: 1165-1173 |
| Arterial vasodilatation | PAR2 Knock Out mice | McGuire et al. Br J Pharmacol 202. 135: 155-169 |
| Sepsis and septic shock | PAR2 inhibitor | Personal data |

According to one aspect of the present invention, 1-piperidine propionic acid can be used, in the treatment of pathological conditions related to PAR2, also in order to alleviate its symptoms and/or to treat or prevent one or more adverse reactions associated therewith.

In accordance with one aspect of the present invention, 1-piperidine propionic acid may also be employed for the treatment of hypercytokinemia, such as, for example, drug-induced hypercytokinemia.

In a further embodiment, the present invention relates to 1-piperidine propionic acid for use in the treatment and/or prevention of sepsis and/or septic shock and its complications.

According to one aspect of the present invention, among the above complications can also be included acute kidney injury or acute kidney failure (AKI) and/or chronic kidney disease (CKD) associated with and/or caused by sepsis.

In a further embodiment of the invention, 1-PPA is for use in the treatment and/or prevention of disease caused by SARS-COV2 or other coronavirus infections, by acting as a PAR2 inhibitor and, due to its effect antithrombotic, for the treatment of complications of the disease caused by this virus (COVID 19).

Furthermore, 1-PPA can be advantageously utilized as an effective pain reliever by acting as a PAR2 inhibitor, since PAR2 has been shown to have a significant impact on the induction and maintenance of persistent, inflammatory, neuropathic, and cancer-associated pain, being involved in the mechanisms of pain perception making the nerve endings very sensitive to the painful stimulus.

The treatment or prevention according to the invention comprises one or more administrations of 1-PPA or pharmaceutical compositions comprising 1-PPA or a salt thereof as the sole active ingredient or in combination with one or more further active ingredients in therapeutically effective doses at a individual who needs it.

Said 1-PPA can be administered in association (concomitant or sequential) with one or more further active principles.

These active ingredients can be active ingredients commonly used in the therapy of the pathologies described in the present description. In a particular embodiment, these active ingredients are anticancer substances. In a particular embodiment such anticancer substances are EGFR inhibitors.

According to one aspect of the present invention, the subjects that can be subjected to a treatment with 1-PPA are in particular subjects for which at least one pathological condition related to PAR2 has been diagnosed. In particular, said condition can be selected from Graft Versus Host Disease (GVHD), Respiratory Distress Syndrome (ARDS), Sepsis, Septic Shock, Ebola, Avian Influenza, COVID-19 Disease, Smallpox, MERS, SARS, Syndrome from systemic inflammatory response, hantavirus pulmonary syndrome, lymphohistiocytosis haemophagocytic disease, tumors that do not overexpress SerpinB3, rheumatoid and inflammatory arthritis, osteoarthritis, posttraumatic osteoarthritis, endothelial dysfunction, acute lung injury (ALI, Acute Lung Injury), Chron's disease, neurodegenerative diseases, Alzheimer's, Parkinson's Multiple sclerosis, ALS, thrombosis caused by an inflammatory event, hyperalgesia, oedema, asthma, respiratory allergies, inflammatory pain, post-surgery pain, neuropathic pain, fracture pain, osteoporotic fracture pain, bone cancer pain or pain gout, irritable bowel syndrome pain, inflammatory bowel pain, ulcerative colitis, cancer, tumors resistant to EGFR inhibitor treatment, inflammatory brain disease, pancreatitis, asthma, metabolic heart disease, atherosclerosis, proliferative retinitis, arterial vasodilatation, pain visceral, neoplastic pain, migraine, cancer pain.

According to one aspect of the invention, 1-piperidine propionic acid may be substituted or used in combination with one or more pharmaceutically acceptable salts thereof.

The dosage of 1-PPA can be established by the doctor on the basis of the weight, state of health, gender and age of the subject to be treated, as well as modified over time on the basis of the subject's response to the therapeutic treatment.

In one embodiment, this active ingredient can be administered in a unit dosage of between 0.01 mg and 10 mg, preferably between 0.05 mg and 5 mg, even more preferably between 0.1 mg and 1 mg.

According to one aspect of the present invention, 1-PPA can be administered in particular in a dosage effective in causing an improvement or even a non-aggravation (that is, keeping the pathological condition stable) of the pathological condition being treated.

Also object of the invention is a pharmaceutical composition for use in the treatment or prevention or to assist the treatment or prevention of PAR2-related pathological conditions, comprising 1-piperidine propionic acid or a salt thereof and at least one pharmaceutically acceptable carrier.

By way of example, the composition according to the present description may comprise from 0.01 mg to 10 mg, preferably from 0.05 mg to 5 mg, even more preferably from 0.1 mg to 1 mg of 1-piperidine propionic acid as dosage unit and one or more doses per day (or fractions of doses) of said composition can be administered to the patient. Alternatively, the composition can be suitably dosed according to the opinion of the attending physician, for example on the basis of the severity of the disorder and of the individual response.

In one embodiment, the active ingredient 1-PPA or a salt thereof can be conveyed by means of delivery systems commonly used by those skilled in the art in such a way as to be able to reach preferential target tissues/sites, therefore, the invention also relates to a composition pharmaceutical for use in the treatment or prevention or to assist the treatment or prevention of PAR2-related pathological conditions as defined and detailed herein, wherein said 1-PPA or a salt thereof is inserted into a suitable pharmaceutical delivery system. A non-limiting example of such a delivery system includes liposomes, micelles, nanoparticles, biodegradable polymers, lipoproteins, vesicles, nanospheres, hydrogels or the like.

The composition according to the invention can be a composition for parenteral, oral, nasal, aerosol, systemic administration and can be formulated in the form of a suspension, emulsion, spray, granulate, powder, solution, capsule, tablet, tablet, lyophilisate, pill, intramuscular or intravenous injection according to conventional pharmaceutical formulation techniques with suitable excipients, carriers, preservatives, diluents and the like. Further components for the formulation of the pharmaceutical composition can be selected from excipients or technological adjuvants used in the common pharmaceutical, cosmetic or food industry practice. The excipients used may belong to the categories of diluents, solubilizers, disintegrating agents, binders, lubricants, surfactants, flow agents and non-stick agents.

In one embodiment, the composition may also contain flavourings, coloring agents and preservatives commonly used in the pharmaceutical industry.

In one embodiment the composition as described herein, in any of the embodiments indicated above, may be in the form of a pharmaceutical composition, i.e. comprise pharmaceutical grade ingredients, or may be or be incorporated into a medical device as hereinafter described in greater detail. In accordance with the invention experiments on the activity of 1-PPA have been conducted and the experiments carried out by the authors of the invention have demonstrated that very low concentrations of 1-PPA (1 ng/ml) are capable of inhibiting the synthesis of PAR2 in certain cell lines under PAR2 overexpression pathological conditions (e.g. THP-1 line and HepG2 line).

Also disclosed herein is a therapeutic method for the treatment or prevention of, or to assist in the treatment or prevention of PAR2-related pathological conditions comprising the administration of 1-piperidine propionic acid or a pharmaceutical composition comprising it in a dosage therapeutically effective to a patient who needs it.

In one embodiment, said pathological conditions are selected from: respiratory distress syndrome (ARDS), sepsis, septic shock, Ebola, avian influenza, COVID-19 disease, smallpox, MERS, SARS, systemic inflammatory response syndrome, pulmonary hantavirus, lymphohistiocytosis haemophagocytic disease, tumors that do not overexpress SerpinB3, rheumatoid and inflammatory arthritis, osteoarthritis, posttraumatic osteoarthritis, endothelial dysfunction, acute lung injury (ALI, Acute Lung Injury), Chron's disease, neurodegenerative diseases, Alzheimer's, Parkinson's, Multiple Sclerosis, ALS, thrombosis caused by an inflammatory event, hyperalgesia, edema, asthma, respiratory allergies, inflammatory pain, pain after surgery, pain from a fracture, pain from osteoporotic fracture, bone cancer pain or joint pain from gout, irritable bowel syndrome pain, inflammatory bowel pain, ulcerative colitis, tumors resistant to treatment with EGFR inhibitors, inflammatory brain disease, pancreatitis, asthma, metabolic heart disease, atherosclerosis, proliferative retinitis, arterial vasodilatation, visceral pain, neoplastic pain, migraine, cancer pain.

The administration of 1-PPA can be repeated several times a day and the association or composition can already be supplied in the form of single doses or with a single dose dispenser.

For the purposes of the present invention, by therapeutically effective amount is meant an amount of active ingredient (1-PPA) which is effective in therapy, or an amount sufficient to provide a desired therapeutic effect. An amount that is effective in therapy is an amount that produces one or more desired biological activities during the treatment or prevention of a pathological condition. In the present case during the treatment or prevention of a disease (pathological condition) as defined in the present description.

In one embodiment, said 1-piperidine propionic acid being administered in one or more doses, in a concentration ranging from 0.01 mg to 10 mg per unit dose, preferably ranging from 0.05 to 0.5 mg per unit dose, even more preferably ranging from 0.01 and 0.1 mg per unit dose.

According to the invention, the therapeutic method for the treatment or for the prevention, or for assisting the treatment or prevention of pathological conditions related to PAR2 may comprise or consist in the application of the medical device comprising the composition of the invention or the 1-PPA or one of its salts, one or more times a day on the affected part.

Finally, the object of the present invention is the use of 1-piperidine propionic acid or a salt thereof according to the present description and claims, or pharmaceutical composition according to the present description and claims, in particular in vitro as a PAR2 inhibitor.

Furthermore, it is an object of the invention, the use of 1-piperidine propionic acid or a salt thereof as PAR2 inhibitory active ingredient in the preparation of pharmaceutical or cosmetic compositions.

The invention also relates to medical devices such as for example medicated plasters, medicated gauze, medicated bandages, medicated wipes, medicated absorbent pads, medicated diapers, i.e. plasters, gauze, bandages, wipes, tampons or diapers which comprise or are covered at least in part of or are at least partially imbued with the composition of the invention described in the most appropriate form or with 1-PPA or a salt thereof.

The gauzes can be made as fatty gauzes, as can the bandages and plasters using the composition made in the form of an ointment, paste or cream for example. The wipes can be soaked with the composition in the form of an emulsion of oil with water or gel, and the diapers or absorbent pads can be made by inserting the composition into suitable layers known in the art for inserting protective or anti-irritant compositions.

Such products as for example diapers for infants, absorbent pads or commonly “absorbents” for women and diapers for adult incontinence, are commonly used for example in the infant sector, in the female sector and in the geriatric sector and the technician of the sector will know where to insert the composition described here and which embodiment is the most suitable without the need for particular teachings and relying solely on conventional techniques in the sector.

These devices will be applicable on the part to be treated and/or to be protected for preventive purposes.

All the experiments reported in this description are carried out on animals from breeding within the University of Padua, bred in compliance with the provisions of Directive 2010/63/EU on the protection of animals used for scientific purposes.

### EXAMPLES

### Example 1. Inhibition of the Membrane Protease Receptor Activated Receptor-2 by 1-Piperidin Propionic Acid

In order to evaluate the effect of 1-Piperidin Propionic acid on the transcriptional activity of the membrane receptor Protease Activated Receptor-2 (PAR2), monocyte-derived cell line THP-1 was used. The cells were incubated with LPS, known to induce PAR2 expression in this cell compartment, at a concentration of 1 ng/ml, or with medium alone, as a control, for 2 hours with 1-PPA at a concentration of 1 ng/ml and 100 ng/ml. In further experiments transfected HepG2 cells were used transiently with plasmid containing the human PAR2 sequence by Lipofectamy. These cells were incubated with medium alone, as a control, or with increasing concentrations of 1-PPA, as described above. Cell extracts from both lines were then subjected to extraction of cellular RNA which was reverse transcribed into cDNA and amplified by qPCR.

The primer sequences used in the study are shown in the following table:

TABLE 1

| Gene | First sense | Antisense primer |
|---|---|---|
| 45S | SEQ ID 1 | SEQ ID 2 |
| Gene ID: | 5′-CGGCTACCACATCCAAGGAA-3′ | 5′-GCTGGAATTACCGCGGGCT-3′ |
| 100861532 | | |
| PAR2 | SEQ ID 3 | SEQ ID 4 |
| Gene ID | 5′-GCTAGCAGCCTCTCTCTCCT-3′ | 5′-GTGGGATGTGCCATCAACCT-3′ |
| 109447 | | |
| TNF-α | SEQ ID 5 | SEQ ID 6 |
| Gene ID: | 5′-AACCTCCTCTCTGCCATCAA-3′ | 5′-GGAAGACCCCTCCCAGATAG-3′ |
| 7124 | | |
| IL-1β | SEQ ID 7 | SEQ ID 8 |
| Gene ID: | 5′-TGAAAGCTCTCCACCTCCAG-3′ | 5′-CACGCAGGACAGGTACAGAT-3′ |
| 3553 | | |

As documented in FIG. 1A, PAR2 transcription is dose-dependently inhibited by PPA, whereas PAR2 expression was found to be markedly increased after incubation with LPS, and addition of 1-PPA induced a marked reduction of its expression, suggesting that the biological action of this compound occurs at the transcriptional level, in particular through the inhibition of the synthesis of the membrane receptor PAR2. Similar results were also obtained in transfected HepG2 cells transiently to overexpress PAR2. In fact, FIG. 1B shows the effective inhibition of PAR2 mRNA by 1-PPA in a dose-dependent manner.

### Example 2. 1-PPA Interacts Sterically and at the Protein Level with PAR2

To evaluate the possible steric interaction between PAR2 and PPA, the structure of PAR2 combined with its known inhibitor (AZ8838) was used as a model for a Molecular Docking, represented in FIG. 2A. The control inhibitor compound (AZ8838) positions itself in the space between the transmembrane helices of the PAR2 receptor and mainly interacts with helices 1-2-7 acting as an ANTAGONIST.

Using this information for the Molecular Docking between the structure of PAR2 and 1-PPA, it has been documented that the two molecules interact in a similar way to that of PAR and AZ8838, as documented in FIG. 2B, from which it is possible to appreciate in black also the various conformations that 1-PPA can assume overall. This conformation was identified as level class 0, which is the highest scoring and most reliable analysis. FIG. 2C shows the result of the Cellular Thermal Shift Assay (CETSA) test and of the Western blot which demonstrate how the presence of PPA determines a thermal stability of the PAR2 protein. In particular, HepG2 cells have been genetically modified to overexpress transiently PAR 2 using cDNA as expression plasmid 3.1. The soluble fraction of the cell extracts was then subjected to the CETSA test, in the presence or absence of 1-PPA, according to the protocol. The samples were also studied by Wester blot using an anti HA-tag PAR 2 monoclonal antibody.

### Example 3. PPA Inhibits Sepsis-Induced Inflammatory Cytokines

Given that PAR2 is deeply implicated in the induction of proinflammatory cytokines, the ability of PPA in the inhibition of proinflammatory cytokines in a context simulating bacterial sepsis was evaluated. In FIGS. 3A and 3B the THP-1 cell line of monocytic origin was used, incubated with LPS (1 mg/ml), which in example 1 had induced an increase in the synthesis of endogenous PAR2. Cells were simultaneously treated with 1-PPA at a concentration of 1 ng/ml or 100 ng/ml or medium alone as control and incubated for 1 or 4 hours. The cell extracts were used to analyze the transcriptional expression of the cytokines TNF-α and IL-1β by RT-PCR. The results confirmed that LPS is able to markedly increase the synthesis of proinflammatory cytokines and that this effect is effectively blocked by the addition of 1-PPA in a dose-dependent manner.

It is important to note how the inhibitory effect is achieved even at very low concentrations of compound (1 ng/ml), in support of its possible double inhibitory effect, both steric and transcriptional.

It should be noted that, unlike the behavior of TNF-α, which has a very early response, the induction of IL-1β by LPS persists over time and the lowest 1-PPA concentration (1 ng/ml) is still more effective in controlling the synthesis of this proinflammatory cytokine.

### Example 4. 1-PPA and Platelet Activation

The process of activation of platelet aggregation, which leads to thrombus formation, is mediated by members of the “protease” family. Activated Receptors” (PAR), in particular PAR 1, PAR 2 and PAR 4.

The analysis of platelet function can be performed by the Multiplate® analyzer (Roche Diagnostics) which is a method based on impedance aggregometry and can be used to measure platelet function in whole blood in the presence or absence of inhibitors. In particular the TRAP test is an assay for the in vitro quantitative determination of platelet function triggered by thrombin receptor activating peptide-6 (TRAP-6) which is a potent platelet activator and stimulates platelet aggregation through the thrombin receptor PAR-1, this peptide has also been found capable of activating PAR2 (Cas #AS-24190-25, CliniScience), which can be activated by thrombin in the presence of thrombomodulin (Heuberger et al, Thrombosis Research 2019, 177:91-101).

Multiplate® Analyzer, Roche Diagnostics) was used, where the blood sample was analyzed alone or in presence of increasing concentrations of 1-PPA.

As shown in FIG. 4, 1-PPA proved capable of inhibiting platelet aggregation in a dose-dependent manner, with excellent reproducibility in the experiments carried out.

These results demonstrate that 1-PPA is able to inhibit PAR2-induced pro-coagulant activity.

### Example 5. 1-PPA Reduces Nociceptive Perception of Pain

To evaluate the inhibitory capacity of 1-PPA in pain perception, notoriously mediated by PAR2 as a receptor of peripheral nerve endings (Andrea S. Rothmeier & Wolfram Ruf. Semin Immunopathol, 2012; 34:133-149), an experimental model of inflammatory arthritis was used. In particular, a mouse model of gout was used by injecting microcrystals of uric acid subcutaneously into the plantar surface of the hind legs of mice (C57BL6J), followed by injection of 1-PPA at different concentrations in one hind leg, using the contralateral leg as a control. After a few hours, the microcrystal treatment induces swelling and edema at the injection site which reaches a peak of inflammation and pain at 48 hours and then resolves spontaneously. FIG. 5 shows how the paw of mice injected with 1-PPA treated urate microcrystals is visually less edematous and reddened than the paw of mice not treated with 1-PPA and these data are confirmed by measuring the circumference of the paws in different animals. treated.

To evaluate the intensity of pain, the electronic von Frey test was used, which involves recording the intensity of the stimulus that evokes a withdrawal reflex, after the application of the pain stimulus to the plantar surface of the hind leg. The right graph documents how mice treated with 1-PPA need a significantly greater stimulus to induce the withdrawal reflex, compared to mice injected only with microcrystals.

### Example 6. In Vivo Experiments Using Models of Systemic Sepsis in Animals in the Presence or Absence of 1-PPA

In vivo experiments are underway on models of systemic sepsis, in order to validate the protective effect of 1-PPA against the development of cytokine storm and septic shock with multiple organ failure, using C57BL/6 J mice. The first part of the study consists in the induction of sepsis through LPS and experimental treatment with 1-PPA.

The intraperitoneal administration with LPS (about 10 mg/kg, L4130, Sigma-Aldrich) associated with 1-PPA or alternatively with PBS, was carried out under anesthesia (Sevorane at 4% in medical oxygen) through the use of Fluovac (Harvard Instruments), dedicated for the anesthesia of small animals.

All treated animals were sacrificed 8 hours after LPS injection (considered time 0), if they did not die during the experimental phase. The time interval of 8 hours is considered the optimal interval for observing the onset of scientifically quantifiable sepsis in mice, and for evaluating the efficacy of a treatment aimed at inhibiting it by administering 1-PPA.

The proximity of the experimental endpoint was carefully chosen in order to minimize the level of suffering of the animal.

Before proceeding with the sacrifice, the animals are anesthetized by deep gaseous anesthesia (4% Sevorane in Fluovac). Immediately after the sacrifice, an intraperitoneal lavage was performed with 2 ml of PBS. Subsequently, any exudate present is aspirated together with the washing solution to evaluate the presence of chemotactic factors, the leukocyte count and for the subsequent study of the expression of various inflammatory factors.

Blood samples are also collected and frozen after obtaining plasma for the quantification of plasma levels of classic markers of organ damage, such as urea nitrogen, creatine kinase (MB cardiac isoenzyme), aspartate aminotransferase (AST) and alanine aminotransferase (ALT) by commercially available kits.

At the same time, the liver and spleen are taken, to evaluate the degree of oxidative stress and any organ damage, and kept in liquid nitrogen.

The experimental response to treatment was evaluated by analysis of variance (ANOVA/Kruskall Wallis rank test).

The results obtained documented that treatment with low concentrations of 1-PPA protected mice from LPS-induced sepsis. FIG. 6A illustrates the experimental design and the quantification of the degree of malaise using a score commonly used to determine the condition of the animal, where 0 refers to the normal condition and 5 refers to a pre-terminal condition. A score of 6 was attributed to the death of the animal. FIG. 6B demonstrates that the lowest concentration of 1-PPA (3 μg) is protective and the animals reach a maximum of a score of 2 which shows mild suffering, with no respiratory effort in the animal, while untreated animals progressively worsen and die.

The study also includes the following parts:
1) Induction of acute kidney disease (CKD) through AF (folic acid) and experimental treatment with 1-PPA inhibitor for PAR2
2) Experimental treatment of acute kidney injury in conjunction with septic shock on pre-existing CKD induced by AF (folic acid) in experimental pathological conditions of Intensive Care Unit (ICU).

Preliminary Phase: A telemetric catheter (Data Science Instruments HDX11 models) to measure blood pressure inside the left carotid artery will be inserted 20 days before the procedure for each animal used for each of the three parts of the study. The telemetric catheter allows the monitoring of arterial pressure during all phases of the procedure through the collection of data transmitted to the Physiotel system (Data Science Instruments). The accurate detection of arterial pressure values, through this minimally invasive system that does not interfere with the animal's normal daily activity, allows to have hemodynamic data with respect to the efficacy of the treatment.

### Telemetry Catheter Insertion Procedure

The mouse is placed under surgical anesthesia. As an anesthetic agent, Sevorane is used (4% in the induction phase, 1.5 in the procedure phase in medical oxygen).

The anesthetized mouse is placed in the supine position and is delicately depilated by applying depilatory cream in the neck region. Once the complete lack of reflexes has been ascertained and after cleaning the operating area with Betadine, the left median region of the neck is delicately incised for a length of about 1 cm. At this point, by gently removing both the subcutaneous fat and the exposed muscle bands with microsurgical tweezers, a small tissue pocket is created which will later house the telemetry device. With the aid of the Leika 2000 operating microscope, the left carotid artery is accurately identified within the incision and delicately clamped using a dedicated microsurgical clamp. The sensor of the telemetry catheter is inserted into the artery using a sterile G24 cannula needle. The sensor is secured using Tabotamp wrapped with three turns of 7-0 non-absorbable suture to ensure the sensor is kept in place. The microclamp is released and hemostasis and catheter tightness is observed once arterial blood flow is resumed. The transmission device is housed in the previously created tissue pocket with care not to damage the salivary glands and surrounding tissue. At this point the incision is closed with a 2-0 and 0 non-absorbable suture. Before waking up, a intramuscle dose of tramadol (5 mg/kg) and adequate antibiotic coverage (clarithromycin associated with cefazolin at 1 mg/kg per Three days).

CKD Induction Protocol (Chronic Kidney Disease) Through AF and Experimental Treatment with the 1-PPA Inhibitor for PAR2

Twenty days before the start of the procedures, the animals are inserted the telemetric catheter for the detection of arterial pressure.

A single intraperitoneal injection of FA (dosage of 125 mg/kg) induces renal damage which reproduces the analogous syndrome in man after 30 days. To ensure maximum precision and least distress for the animal, the 1-PPA inoculation is performed by anesthetizing the mouse for a few minutes using deep inhalation surgical anesthesia using 4% Sevorane in medical oxygen administered through a Fluovac device (Harvard Instruments) specifically dedicated to the anesthesia of laboratory rodents.

Treatment with 1-PPA (i.p. injection at a concentration of 0.1 μg/g of body weight) begins from the third day after the induction of renal damage (groups FA+1-PPA). Control groups are treated with PBS (FA+PBS groups).

Renal function is monitored weekly by semiquantitative evaluation of proteinuria using multistix (Siemens). For urine collection, the rat is placed in a carefully cleaned plexiglass tray, in the presence of dedicated environmental enrichment. After 30 days from the induction of the renal damage the animals are sacrificed by cervical dislocation or excess anesthesia. Peripheral blood is collected by intracardiac puncture, and kidneys and liver are sampled in formalin and at −80° C. for subsequent evaluations of IHC and gene expression.

For the groups treated with FA+1-PPA, reached the thirtieth day from the induction of the renal disease, a mean value of proteinuria measured in the urine is expected which corresponds to a moderate residual damage. As regards the control groups (FA+PBS), a significant slowing down of the renal disease is not expected, so on the 30th day the expected result for these animals is proteinuria with values on average higher than 1000 mg/dl in analogy with what observable in the experimental model if renal disease is allowed to progress spontaneously (Howie and Helyer, 1968; Puttermann et al, 1988).

Protocol for the Induction of Sepsis Through LPS and Experimental Treatment with 1-PPA

Twenty days before the start of the procedures, the animals were inserted the telemetric catheter for detecting arterial pressure as described above.

The model of sepsis obtained by single intraperitoneal administration of LPS (lipopolysaccharide) will be used. This procedure allows to study the organ dysfunction following a robust and early systemic inflammatory response involving all the main inflammatory mediators (Moon et al., 2015; Moon et al., 2016).

### Pre-Existing CKD in Experimental Intensive Care Unit (ICU) Pathological Conditions.

Twenty days before the start of the procedures, the animals were inserted the telemetric catheter for detecting arterial pressure as described above.

Renal disease (CKD) is induced with an intra-peritoneal injection of AF (125 mg/kg) as described above.

After 14 days, with already consolidated renal damage, the CLP procedure is surgically performed (Cecal Ligation and Puncture) to induce sepsis and subsequent acute kidney injury.

The aim of this procedure is to validate the efficacy of 1-PPA in an experimental model that accurately reproduces what happens in a patient with pre-existing CKD who incurs sepsis with the consequent acute kidney injury.

dedicated Fluovac device by administering Sevorane (4% induction and 1.5% surgical phase) and treated with Cefazolin (1 mg/kg sc).

After an abdominal laparotomy has been performed and the cecum has been gently isolated, a 4-0 silk thread ligation is performed approximately 8 mm from the caecal end. The anatomical landmark in which to strangle is the one in which the most probable induction of sub-lethal sepsis has been observed (Doy et al, Kidney Inter. 2008).

Once tied, the cecum is punctured superficially with a 21 G flute-point needle and gently squeezed with anatomical tweezers so as to release a fragment of faecal material into the peritoneum. Before proceeding with the surgical suture of the incision and, to reproduce what happens for hospitalized patients in intensive care and suffering from renal disease, the bowels are sprayed with 1 ml of saline solution pre-heated to 37 degrees.

Just before the mouse is awakened, post-surgical analgesia is ensured by intramuscular administration of 5 mg/kg tramadol.

pre-warmed saline solution at 37 degrees and treated with 7 mg/kg of imipenem/cilastatin sodium sc

From the initial moment to the conclusion of the procedures and to the sacrifice (expected after 6 hours) the animal is closely observed and rigorously monitored for the entire duration of the procedure by the personnel involved in the experiment.

### Echo-Cardiographic Evaluation

Close to the end-point and immediately before the sacrifice, an echo-cardiographic evaluation is performed to determine the effect of the experimental treatment with 1-PPA on the main cardiac and hemodynamic parameters in the presence of renal disease and septic shock.

The procedure and the parameters follow what is indicated in the publication of Kalbitz M and colleagues “Complement-induced activation of the cardiac NLRP3 inflammasome in sepsis” Faseb J 2019.

The echo-cardiographic examination is performed using a Vevo 2100 high-resolution echograph dedicated to laboratory rodents (Visualsonics, Canada), using a 40 MHz RMV-707 probe.

After deep surgical anesthesia (4% Sevorane in medical oxygen and 1% in induction phase), the probe positioned on the echo-opaque table is positioned in the mouse in order to acquire the echographic image along the parasternal long axis in order to to acquire the following parameters:
1) Heart rate, LV volume, Vol S, Vol D. From these parameters, stroke volume (SV) and ejection fraction (EF) and cardiac output (PO) are calculated.
2) The correlations between the flow and any pressure gradients are investigated by means of Echo-Doppler by measuring the isovolumetric contraction and relaxation time from the moment of closure of the aortic valve to the opening of the mitral valve. At 6 hours the animal is sacrificed by cervical dislocation or excess anesthesia.

After the sacrifice, intraperitoneal exudate, peripheral blood, kidneys, liver, heart and lungs are collected and sampled for subsequent analysis.

In the peripheral blood the following are quantified: creatinine, blood urea nitrogen, leukocyte count and the main mediators of circulating inflammation.

In the kidney in particular, glomerular damage, the number of glomeruli involved, the leukocyte infiltrate and the degree and type of interstitial fibrosis are evaluated as morphological parameters.

### Example 7. Toxic Studies of 1-Piperidine Propionic Acid

Studies were conducted to evaluate the possible toxicity of the 1-PPA compound in vitro on the HepG2 cell line (code HB8065), by incubating the cells with increasing concentrations of 1-PPA (5-50 g/ml), much higher than those resulted able to inhibit the synthesis of PAR2 and its effects in the previously described in vitro systems. Real-time cell proliferation was monitored using the XCelligence tool (ACEA) and the results were expressed as Cell Index, which expresses the cell number at each point considered. As documented in FIG. 7A,B, PPA did not demonstrate a cellular toxic effect at the concentration of 5 μg/ml, which is at least 50 times higher than the biologically effective dose. In particular, the EC50, which is the compound concentration determining 50% of the maximal effect, calculated for PPA under these experimental pathological conditions, was found to be 22 μg/ml, more than 100 times higher than the biologically effective dose.

The organ damage of the 1-PPA compound in vivo was also evaluated, considering the function of the liver and kidney, the main organs responsible for pharmacological catabolismln detail, the study included 2 groups of CC57/BL6 mice (4 mice/group, age 45 days), inoculated with 1-PPA at concentrations of 0.07 and 0.7 μg/g/week), for a time period of 26 weeks. All animals were fed a normal diet throughout the study. Before starting the 1-PPA inoculation, a basal blood sample was taken from all animals and a second sample was taken from the sacrifice.

At serum level, alanine aminotransferase (ALT) as an indicator of hepatic cytolytic damage, bilirubin as an indicator of hepatic cholestasis, blood urea nitrogen (Blood urea nitrogen, BUN) as an indicator of renal damage were evaluated. The results were expressed as the ratio between the value of the final sampling and that of the basal sampling. As shown in FIG. 7C, in mice fed with a normal diet, doses of 0.07 and 0.7 μg/g/week did not cause significant changes in the values of both liver and kidney damage (<2×normal values). These results show a negligible effect cholestatic and renal function.

1-13. (canceled)

14. A method of treatment or prevention of protease activated receptor 2 (PAR2) related pathological conditions comprising administering a PAR2 inhibitor to a patient in need thereof, wherein said inhibitor is 1-piperidine propionic acid or a salt thereof, and wherein said PAR2 related pathological conditions are selected from:
hantavirus pulmonary syndrome, lymphohistiocytosis haemophagocytic disease, tumors that do not overexpress SerpinB3, rheumatoid and inflammatory arthritis, osteoarthritis, posttraumatic osteoarthritis, endothelial dysfunction, acute lung injury (ALI, Acute Lung Injury), Crohn's disease, Multiple Sclerosis, amyotrophic lateral sclerosis (ALS), thrombosis caused by an inflammatory event, hyperalgesia, edema, asthma, respiratory allergies, inflammatory pain, post-surgery pain, fracture pain, pain from osteoporotic fracture, bone cancer pain or joint pain from gout, irritable bowel syndrome pain, inflammatory bowel pain, ulcerative colitis, tumors resistant to treatment with EGFR inhibitors, inflammatory brain disease, pancreatitis, asthma, metabolic heart disease, atherosclerosis, proliferative retinitis, arterial vasodilatation, visceral pain, neoplastic pain, migraine and cancer pain,
wherein said treatment comprises or consists in the administration of 1-piperidine propionic acid or a salt thereof, or of a pharmaceutical composition comprising as active ingredient, alone or in combination with further active ingredients, 1-piperidine propionic acid or a salt thereof and at least one pharmaceutically acceptable excipient or carrier.

15. The method according to claim 1 wherein said 1-piperidine propionic acid or salt thereof is administered in one or more doses, in a concentration ranging from 0.01 mg to 10 mg per unit dose.

16. The method according to claim 1 wherein said 1-piperidine propionic acid or salt thereof is administered in one or more doses, in a concentration ranging from 0.05 to 0.5 mg per unit dose.

17. The method according to claim 1 wherein said 1-piperidine propionic acid or salt thereof is administered in one or more doses, in a concentration ranging from 0.01 to 0.1 mg per unit dose.

18. The method according to claim 1 wherein said 1-piperidine propionic acid or salt thereof is administered in a delivery system such as a liposome, a micelle, a nanoparticle, a biodegradable polymer, a lipoprotein, a vesicle, a nanosphere.

19. The method according to claim 1 wherein said 1-piperidine propionic acid or salt thereof is administered in the form of a pharmaceutical composition further comprising one or more of a pharmaceutically acceptable stabilizer, and/or a diluent and/or an excipient.

20. The method according to claim 19 wherein said pharmaceutical composition is administered via parenteral, oral, nasal, aerosol, sublingual or systemic administration.

21. The method according to claim 19 wherein said pharmaceutical composition is in the form of a suspension, emulsion, spray, granulate, powder, solution, capsule, tablet, cream, ointment, ointment, lozenge, lyophilisate, pill or injection.

22. A method for inhibiting PAR2 comprising contacting PAR2 with 1-piperidine propionic acid or a salt thereof.
